(19) United States
(12) Patent Application Publication
(10) Pub. No.: US 2008/0279800 A1
(43) Pub. Date: Nov. 13, 2008

(54) AROMACHEMICALS

(75) Inventor: Luca TURIN, London (GB)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Flexitral, Inc., Chantilly, VA (US)

(21) Appl. No.: 12/039,037

(22) Filed: Feb. 28, 2008

Related U.S. Application Data
(60) Provisional application No. 60/891,994, filed on Feb. 28, 2007.

(51) Int. Cl. A61K 8/49; C07D 303/04; C11D 3/50; A61Q 13/00; A61Q 15/00; A01N 25/00; C07D 331/02

(52) U.S. Cl. 424/65; 549/1; 549/512; 549/519; 512/11; 510/103; 514/772; 424/76.1

(57) ABSTRACT

A compound of formula (I):

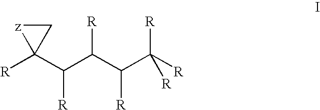

wherein each R independently represents H, methanol or ethanol, and Z represents O or S.

### CROSS REFERENCE INFORMATION

This application claims priority to U.S. Provisional Patent Application No. 60/891,994, filed Feb. 28, 2007, which is fully incorporated herein by reference.

### BACKGROUND OF THE INVENTION

1. Field of the Invention

The present invention relates generally to the field of fragrances. More particularly, the present invention relates to derivatives of certain compounds that have fragrance properties, which are not necessarily shared by the compounds from which they are derived and thus provide perfumes and other articles with properties and advantages not provided by the compound from which they are derived. These derivatives find utility in any and all applications requiring fragrances. The invention also provides mixtures of these derivatives, methods for their preparation and their use as perfume materials for application in a variety of substrates and their use in articles of manufacture and compositions including the derivatives.

2. Technical Background

There are a large number and variety of known fragrances used as ingredients in perfumes and in a varied range of other products. However, many aromachemicals include double bonds and/or other reactive groups that are potentially susceptible to reaction and may result in a limited useful lifetime. Further, many essential oil fragrances have recently been determined to cause allergic reactions, and it is becoming increasingly difficult to bring products containing such fragrances to market. There is also a demand for new fragrances that have novel or improved fragrance profiles and/or other properties that make them particularly useful for use as fragrances.

### SUMMARY OF THE INVENTION

According to one aspect of the invention, there is provided aromachemical compositions. Another aspect of the invention provides for a method for producing aromachemical compositions.

In one embodiment, the invention comprises a compound of formula (I):

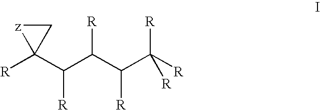

wherein each R independently represents H, methyl or ethyl, and Z represents O or S.

In another embodiment, the invention comprises a process for preparing a compound of formula (I)

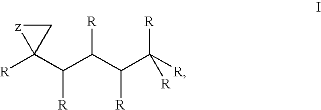

the process comprising converting a compound of formula

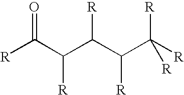

to the compound of formula (I), wherein each R independently represents H, methyl or ethyl, and Z represents O or S.

In yet another embodiment, the invention is to a composition, product, preparation or article having aroma, fragrance or odour releasing characteristics comprising:

an olfactory effective amount of a compound of formula (I):

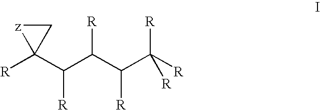

wherein each R independently represents H, methyl or ethyl, and Z represents O or S; and

one or more compounds selected from the group consisting of perfuming ingredients, solvents and adjuvants.

In a particular embodiment, the invention is to a method for treating a substrate to impart a fragrance or fragrance releasing characteristics thereto comprising treating the substrate with an olfactory effective amount of a compound of formula (I):

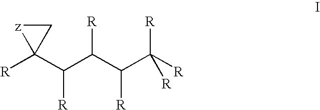

wherein each R independently represents H, methyl or ethyl, and Z represents O or S.

In yet another embodiment, the invention provides for a method to confer, improve, enhance or modify an aroma, fragrance or odour characteristics of a composition, product, preparation or article which comprises adding thereto an olfactory effective amount of a compound of formula (I):

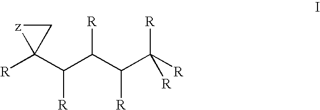

wherein each R independently represents H, methyl or ethyl, and Z represents O or S.

In a particular embodiment, at least one R is methyl. In another, each R is methyl. Preferably, Z is O.

In another particular embodiment, the composition, product or article of the invention is in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

The invention is also directed to the use of the noted compounds as a fragrance.

### DETAILED DESCRIPTION OF THE PREFERRED EMBODIMENTS

The present invention provides compounds of formula (I):

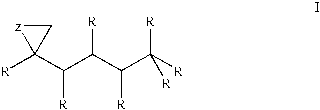

Each R may be the same or different and is selected from H, methyl and ethyl. Preferably, at least one R represents methyl. More preferably each R is methyl. Z represents O or S. Preferably, Z represents O. Particularly preferred compounds of the invention are those in which at least one R is methyl and Z is O.

Non-limiting examples of compounds of formula (I) include the compounds of formula (I′), namely 2-methyl-2-(1,2,3,4,4-pentamethylpentyl)oxirane (shown below). This is the compound of formula (I) in which each R is methyl and Z is O.

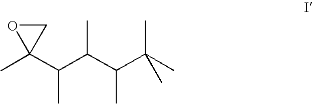

The compounds of the invention typically are also substantially not toxic to humans and animals. For example, 2-methyl-2-(1,2,3,4,4-pentamethylpentyl)oxirane has been found to have an oral LD₅₀ of greater than 2000 mg/kg in rats.

The compounds of the invention may be prepared from any suitable starting material. For example, they can be prepared from the corresponding ketone or aldehyde. In one aspect of the invention, the compounds of the invention can be prepared starting from the corresponding alcohol, via the corresponding ketone or aldehyde. For example, when Z=0, the compounds of the invention may be prepared as depicted below:

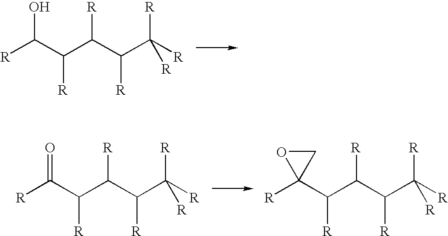

Oxidation of the alcohol (III) to the aldehyde/ketone (II) may be carried out in any suitable manner (see for example, Advanced Organic Chemistry, March, Fourth Edition, pages 1167 to 1171). A preferred group of reagents for converting the alcohol (III) to the aldehyde/ketone (II) is chromium (VI) reagents, including aqueous chromic and sulphuric acid (the Jones Reagent), dipyridine chromium (VI) oxide (Collins' reagent), pyridinium chlorochromate (Corey's reagent) and pyridinium dichromate.

Conversion of the aldehyde/ketone to the epoxide may be carried out in any suitable manner, for example using sulphur ylides (see Advanced Organic Chemistry, March, Fourth Edition, pages 974 to 975). Other methods for producing epoxides are described in March, Fourth Edition, page 1285.

In another aspect, the compounds of the invention in which Z is O can be made in accordance with the following reaction scheme.

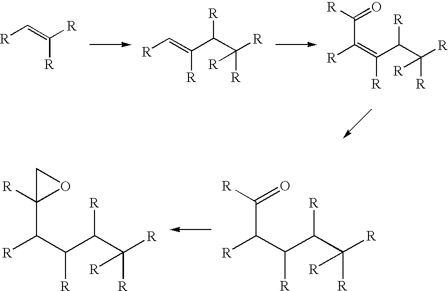

For the compounds of the invention in which each R is O, this reaction scheme can be summarised as follows:

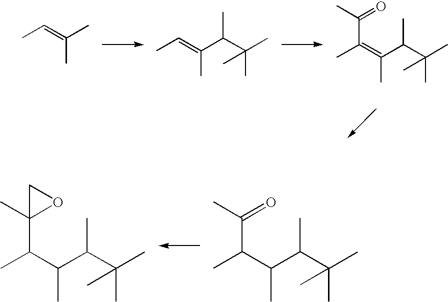

When each R is methyl (Me), a suitable starting material is the alcohol Kohinool®, or 3,4,5,6,6-pentamethyl-2-heptanol, which is commercially available from International Flavours and Fragrances Inc. (www.iff.com). Oxidation of the alcohol to the corresponding ketone followed by conversion to the epoxide gives 2-methyl-2-(1,2,3,4,4-pentamethylpentyl)oxirane.

In summary, the invention provides a process for preparing the compounds of the invention, the process comprising converting a compound of formula

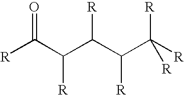

to a compound of formula (I)

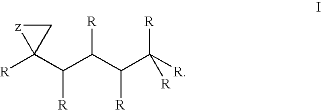

By way of example, when Z=O, converting means epoxidizing.

The process may also comprise oxidising a compound of formula

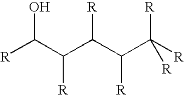

to produce the compound of formulas

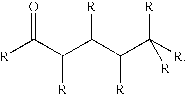

The compounds of the invention in which Z is S can be prepared by any suitable method for producing thiiranes. For example, the compounds of the invention in which Z is O can be converted to the corresponding thiirane. This can be done using any suitable known method. For example, a compound of the invention in which z is O can be reacted with thiourea. Methods for producing thiiranes (epiulfides) are described in March, Fourth Edition, pages 408, 409 and 1285.

Compounds of the invention may exhibit tautomerism. All tautomeric forms and mixtures thereof are included within the scope of the invention.

Compounds of the invention may contain one or more asymmetric carbon atoms and may therefore exhibit optical and/or diastereoisomerism. Diastereoisomers may be separated using conventional techniques, e.g. chromatography or fractional crystallisation. The compounds of the invention may be used as a racemic mixture of stereoisomer or may be separated into individual isomers which may then be used separately are in pre-selected ratios. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst, all under conditions known to the skilled person. All stereoisomers and mixtures thereof are included within the scope of the invention.

The present invention provides for the use of the compounds of the invention and mixtures thereof as a fragrance.

The present invention also provides compositions, products, preparations or articles containing a compound or mixture of compounds of the invention.

A method to confer, improve, enhance or modify the aroma, fragrance or odor characteristics of compositions, products, preparations or articles which comprises adding thereto an aroma, fragrance or odor effective amount of a compound or mixture of compounds of the invention is provided.

The compounds of the invention can be included in virtually any article of manufacture that can include fragrance compounds. Examples include hypochlorite (bleach) compositions, detergents and perfumes. The compounds of the invention can be used in applications like soaps, shampoos, denture cleanser tablets, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they may be used in other current uses in perfumery, namely the perfuming of soaps and shower gels, hygiene or hair-care products, as well as of body deodorants, air fresheners and cosmetic preparations, and in fine perfumery, namely in perfumes and colognes.

The compounds of the invention are particularly suitable for use in fine fragrances and in personal care products.

The compounds of the invention can be used as perfuming ingredients, as single compounds or as mixtures thereof. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants.

Perfuming co-ingredients include one or more of alcohols, aldehydes, ketones, esters, ethers, acetates, nitrites, terpene hydrocarbons, sulfur- and nitrogen containing heterocyclic compounds, as well as essential oils of natural or synthetic origin.

The proportions in which the compounds of the invention can be incorporated in the various products vary as appropriate. These values depend on the nature of the article or product that one desires to perfume and the odor effect searched for, as well as on the nature of the co-ingredients in a given composition when the compounds are used in admixture with perfuming co-ingredients, solvents or adjuvants.

The fragrance composition of this invention contains an olfactory (e.g., aroma, fragrance or odor) effective amount of the fragrance enhancing composition. According to this invention, olfactory effective amount refers to the amount of compound in fragrance compositions the individual component will contribute to its particular olfactory characteristics. The olfactory effect of the fragrance compositions will be the sum of the effects of each of the fragrance ingredients. Thus, the compounds of the invention can be used to alter the aroma characteristics of the fragrance composition, or by modifying the olfactory reaction contributed by another ingredient in the composition. The amount will vary depending on many factors including other ingredients, their relative amounts and the effect that is desired.

In one embodiment, the compound of the invention is present in the fragrance composition in an amount of at least 0.005 percent by weight, based on total weight of the composition. Preferably, the compound of the invention is present in the fragrance composition in an amount of at least 0.05 percent by weight, more preferably at least 0.1 weight percent, and most preferably at least 1 weight percent, based on total weight of the composition.

In another embodiment, the invention is a perfumed article that comprises from about 0.005 weight percent to about 10 weight percent, preferably from about 0.5 weight percent to about 8 percent, and most preferably from about 1 weight percent to about 7 weight percent, based on total weight of the perfumed article.

In various preferred embodiments, other agents are used in conjunction with the fragrance. Non-limiting examples include surfactants, emulsifiers, and fragrance-encapsulating polymers.

In certain fragrance compositions, the compound of the invention should be sufficiently high so as to provide the appropriate olfactory effective amount of the compound. In one embodiment, the fragrance composition contains up to 70 weight percent of the olfactory enhancing compound, based on total weight of the composition.

In another embodiment, the fragrance composition contains up to 60 weight percent, in another up to 50 weight percent, and in another up to 25 weight percent, based on total weight of the composition.

In another embodiment of the invention, the compounds of the invention are present at concentrations from about 0.001% to about 60%, in another from about 0.01% to about 30%, by weight relative to the weight of the composition, product or article in which they are incorporated. It will be appreciated that the amount by weight of a compound of the invention in a particular composition or product will depend on the nature of the composition. In one particular embodiment, the composition is a washing powder that contains from 0.001% by weight to 1% by weight of a compound of the invention. In another embodiment, the composition is a fine fragrance that contains from 20% by weight to 70% by weight of a compound of the invention.

In yet another embodiment of the invention, the compounds are used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts.

In addition to the compounds of the invention, the compositions can include at least one detersive surfactant and optionally, one or more other detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein, preferably in an amount of from about 0.5% to about 90 wt %, by weight, include the conventional C1-18 alkyl benzene sulfonates (“LAS”) and primary, branch-chain and random C10-20 alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities.

In one embodiment, the composition that contains one or more compounds of the invention is a gel, preferably a hydrogel, that contains an olfactory effective amount at least one of the compounds of the invention. The gel can contain up to 90% by weight of the compound of the invention. The gels are preferably prepared from a polymer having a hydroxy (lower alkoxy) 2-alkeneoate, a hydroxy (lower alkoxy) lower alkyl 2-alkeneoate, or a hydroxy poly (lower alkoxy) lower alkyl 2-alkeneoate and a polyethylenically unsaturated crosslinking agent. These materials preferably have continuous slow release properties, i.e. they release the fragrance component continuously over a long period of time.

### EXAMPLES

The present invention is illustrated by the following non-limiting examples.

### Example 1

### Synthesis of 3,4,5,6,6-pentamethyl-2-heptanone

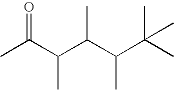

To a stirred solution of pyridinium dichromate in dichloromethane at 25° C. was added a solution of 3,4,5,6,6-pentamethyl-2-heptanol in dichloromethane. The reaction was monitored by TLC and when the alcohol had been consumed celite and diethyl ether were added. The suspension was filtered through a pad of celite and the filtrate concentrated to yield the ketone. The ketone may be further purified by passing through silica gel in a suitable eluant.

### Example 2

### Synthesis of 2-methyl-2-(1,2,3,4,4-pentamethylpentyl)oxirane

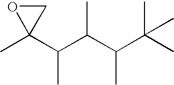

Trimethylsulfonium bromide (1 equiv) and potassium hydroxide (2 equiv) were stirred in a mixture of acetonitrile and water at 60° C. A solution of the ketone in acetonitrile was added and the reaction monitored. Once complete the solution was filtered and the filtrate concentrated to remove the acetonitrile. The solution was partitioned between water and petroleum ether, the organic layer separated and concentrated to yield the crude epoxide. Further purification may be achieved by chromatography on silica gel using a suitable eluant.

This compound had an intense sweet amber odour.

Toxicology tests were conducted on the product and 2-methyl-2-(1,2,3,4,4-pentamethylpentyl)oxirane has been found to have an oral LD₅₀ of greater than 2000 mg/kg in rats.

### Example 3

### Synthesis of 2-methyl-2-(1,2,3,4,4-pentylmethylpentyl)thiirane

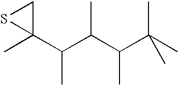

An equimolar mixture of the epoxide made in the step above and thiourea in ethanol (340 mL) and water (270 mL) was stirred at room temperature overnight. The solvent was evaporated and the residue extracted using petroleum ether. The solvent was evaporated to yield the product.

### Example 4

### Incorporation of 2-methyl-2-(1,2,3,4,4-pentamethylpentyl)oxirane (Kohinox) into a fragrance formulation (Ambery floral composition)

A fragrance formulation according to the following Table is prepared

TABLE

| Compound | grams |
|---|---|
| Phenylethyl alcohol | 150 |
| Calone | 1 |
| Citronellol | 20 |
| Galaxolide | 200 |
| Methyl Dihydro Jasmonate | 150 |
| Helional | 15 |
| Beta Ionone | 15 |
| Kohinox | 150 |
| Lilial | 130 |
| Linalool | 49 |
| Melonal | 3 |

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A compound of formula (I): 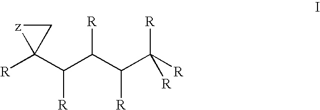
wherein each R independently represents H, methyl or ethyl, and Z represents O or S.

2. The compound of claim 1, wherein at least one R is methyl.

3. The compound of claim 2, wherein each R is methyl.

4. The compound of claim 1, wherein Z is O.

5. The compound of claim 3, wherein Z is O.

6. A process for preparing a compound of formula (I) 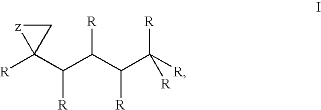
the process comprising converting a compound of formula 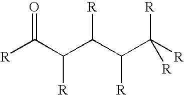
to the compound of formula (I), wherein each R independently represents H, methyl or ethyl, and Z represents O or S.

7. The process of claim 6, wherein the compound of formula 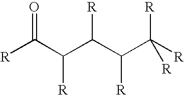
is obtained or has been obtained by oxidizing a compound of formula

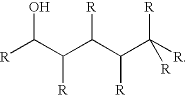

8. The process of claim 7, wherein the oxidation is carried out using a chromium (IV) reagent.

9. The process of claim 6, for producing a compound of formula (I) in which Z=0, which process comprises reacting the compound of formula 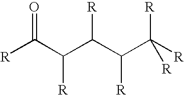
with a sulphur ylide.

10. A method for treating a substrate to impart a fragrance or fragrance releasing characteristics thereto comprising treating the substrate with an olfactory effective amount of a compound of formula (I): 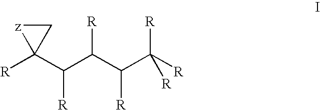
wherein each R independently represents H, methyl or ethyl, and Z represents O or S.

11. The method of claim 10, wherein at least one R is methyl.

12. The method of claim 11, wherein each R is methyl.

13. The method of claim 10, wherein Z is O.

14. The method of claim 12, wherein Z is O.

15. A composition, product, preparation or article having aroma, fragrance or odour releasing characteristics comprising:
an olfactory effective amount of a compound of formula (I): 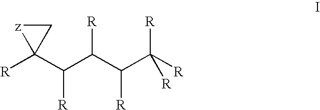
wherein each R independently represents H, methyl or ethyl, and Z represents O or S; and
one or more compounds selected from the group consisting of perfuming ingredients, solvents and adjuvants.

16. The composition, product, preparation or article of claim 15, wherein at least one R is methyl.

17. The composition, product, preparation or article of claim 16, wherein each R is methyl.

18. The composition, product, preparation or article of claim 15, wherein Z is O.

19. The composition, product, preparation or article of claim 17, wherein Z is O.

20. The composition, product, preparation or article of claim 15, wherein the composition, product or article is in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

21. A method to confer, improve, enhance or modify an aroma, fragrance or odour characteristics of a composition, product, preparation or article which comprises adding thereto an olfactory effective amount of a compound of formula (I): 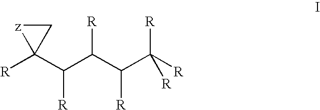
wherein each R independently represents H, methyl or ethyl, and Z represents O or S.

22. The method of claim 21, wherein at least one R is methyl.

23. The method of claim 22, wherein each R is methyl.

24. The method of to claim 21, wherein Z is O.

25. The method of claim 23, wherein Z is O.

26. The method of claim 21, wherein the composition, product, preparation or article is in the form of a perfume, a body odorant, deodorant or antiperspirant, a detergent, a bleach product or a disinfectant.
